(19) United States
(12) Patent Application Publication
(10) Pub. No.: US 2026/0241173 A1
(43) Pub. Date: Aug. 20, 2026

(54) PROGRAMMING OF NEURAL STIMULATION THERAPY WITH MULTIPLE STIMULATION SETS

(71) Applicant: Saluda Medical Pty Limited, Macquarie Park, New South Wales (AU)

(72) Inventors: Dean Michael Karantonis, Macquarie Park (AU); Ian Cameron Gould, Macquarie Park (AU); Daniel John Parker, Macquarie Park (AU); Peter Scott Vallack Single, Macquarie Park (AU); Matthew Marlon Williams, Macquarie Park (AU); Zubin Zarir Nanavati, Macquarie Park (AU)

(21) Appl. No.: 18/870,917

(22) PCT Filed: Jun. 2, 2023
(86) PCT No.: PCT/AU2023/050481

§ 371 Date: Dec. 2, 2024

(30) Foreign Application Priority Data
Jun. 2, 2022 (AU) 2022901521
Mar. 10, 2023 (AU) 2023900647

(51) Int. Cl. A61N 1/36; A61N 1/05

(52) U.S. Cl. CPC A61N 1/36139; A61N 1/0529; A61N 1/36067; A61N 1/36071; A61N 1/3615; A61N 1/36185

(57) ABSTRACT

Disclosed is a neurostimulation system comprising a multiple-stimset closed-loop neurostimulation device, and a processor. The multiple-stimset closed-loop neurostimulation device is configured to controllably deliver neural stimuli according to a plurality of stimulation sets to a neural pathway of a patient so as to maintain a neural response intensity for an applied stimulation set of the plurality of stimulation sets at a corresponding target value. The processor is configured to: instruct the multiple-stimset closed-loop neurostimulation device to deliver a plurality of neural stimuli according to a first stimulation set of the plurality of stimulation sets; receive a captured signal window corresponding to each delivered neural stimulus; measure a characteristic of an evoked neural response in each captured signal window; and determine a quality metric for the first stimulation set from the measured characteristics.

The present application claims priority from Australian Provisional Patent Applications Nos. 2022901521 filed on 2 Jun. 2022 and 2023900647 filed 10 Mar. 2023, the contents of which are incorporated herein by reference in their entirety.

### TECHNICAL FIELD

The present invention relates to neural stimulation therapy and in particular to programming neural stimulation therapy with multiple stimulation sets.

### BACKGROUND OF THE INVENTION

There are a range of situations in which it is desirable to apply neural stimuli in order to alter neural function, a process known as neuromodulation. For example, neuromodulation is used to treat a variety of disorders including chronic neuropathic pain, Parkinson's disease, and migraine. A neuromodulation device applies an electrical pulse (stimulus) to neural tissue (fibres, or neurons) in order to generate a therapeutic effect. In general, the electrical stimulus generated by a neuromodulation device evokes a neural response known as an action potential in a neural fibre which then has either an inhibitory or excitatory effect. Inhibitory effects can be used to modulate an undesired process such as the transmission of pain, or excitatory effects may be used to cause a desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a device typically comprises an implanted electrical pulse generator, and a power source such as a battery that may be transcutaneously rechargeable by wireless means, such as inductive transfer. An electrode array is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in orthodromic (in afferent fibres this means towards the head, or rostral) and antidromic (in afferent fibres this means towards the cauda, or caudal) directions. Action potentials propagating along Aβ (A-beta) fibres being stimulated in this way inhibit the transmission of pain from a region of the body innervated by the target neural fibres (the dermatome) to the brain. To sustain the pain relief effects, stimuli are applied repeatedly, for example at a frequency in the range of 30 Hz-100 Hz.

For effective and comfortable neuromodulation, it is necessary to maintain stimulus intensity above a recruitment threshold. Stimuli below the recruitment threshold will fail to recruit sufficient neurons to generate action potentials with a therapeutic effect. In almost all neuromodulation applications, response from a single class of fibre is desired, but the stimulus waveforms employed can evoke action potentials in other classes of fibres which cause unwanted side effects. In pain relief, it is therefore desirable to apply stimuli with intensity below a discomfort threshold, above which uncomfortable or painful percepts arise due to over-recruitment of Aβ fibres. When recruitment is too large, Aβ fibres produce uncomfortable sensations. Stimulation at high intensity may even recruit Aδ (A-delta) fibres, which are sensory nerve fibres associated with acute pain, cold and heat sensation. It is therefore desirable to maintain stimulus intensity within a therapeutic range between the recruitment threshold and the discomfort threshold.

The task of maintaining appropriate neural recruitment is made more difficult by electrode migration (change in position over time) and/or postural changes of the implant recipient (patient), either of which can significantly alter the neural recruitment arising from a given stimulus, and therefore the therapeutic range. There is room in the epidural space for the electrode array to move, and such array movement from migration or posture change alters the electrode-to-fibre distance and thus the recruitment efficacy of a given stimulus. Moreover, the spinal cord itself can move within the cerebrospinal fluid (CSF) with respect to the dura. During postural changes, the amount of CSF and/or the distance between the spinal cord and the electrode can change significantly. This effect is so large that postural changes alone can cause a previously comfortable and effective stimulus regime to become either ineffectual or painful.

Attempts have been made to address such problems by way of feedback or closed-loop control, such as using the methods set forth in International Patent Publication No. WO2012/155188 by the present applicant. Feedback control seeks to compensate for relative nerve/electrode movement by controlling the intensity of the delivered stimuli so as to maintain a substantially constant neural recruitment. The intensity of a neural response evoked by a stimulus may be used as a feedback variable representative of the amount of neural recruitment. A signal representative of the neural response may be sensed by a measurement electrode in electrical communication with the recruited neural fibres, and processed to obtain the feedback variable. Based on the response intensity, the intensity of the applied stimulus may be adjusted to maintain the response intensity within a therapeutic range.

It is therefore desirable to accurately measure the intensity and other characteristics of a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be measured at a measurement electrode as a positive peak potential, then a negative peak, followed by a second positive peak. This morphology is caused by the region of activation passing the measurement electrode as the action potentials propagate along the individual fibres.

Approaches proposed for obtaining a neural response measurement are described by the present applicant in International Patent Publication No. WO2012/155183, the content of which is incorporated herein by reference.

However, neural response measurement can be a difficult task as a neural response component in the sensed signal will typically have a maximum amplitude in the range of microvolts. In contrast, a stimulus applied to evoke the response is typically several volts, and manifests in the sensed signal as crosstalk of that magnitude. Moreover, stimulus generally results in electrode artefact, which manifests in the sensed signal as a decaying output of the order of several millivolts after the end of the stimulus. As the neural response can be contemporaneous with the stimulus crosstalk and/or the stimulus artefact, neural response measurements present a difficult challenge of measurement amplifier design. For example, to resolve a 10 μV ECAP with 1 μV resolution in the presence of stimulus crosstalk of 5 V requires an amplifier with a dynamic range of 134 dB, which is impractical in implantable devices. In practice, many non-ideal aspects of a circuit lead to artefact, and as these aspects mostly result a time-decaying artefact waveform of positive or negative polarity, their identification and elimination can be laborious.

Evoked neural responses are less difficult to detect when they appear later in time than the artefact, or when the signal-to-noise ratio is sufficiently high. The artefact is often restricted to a time of 1-2 ms after the stimulus and so, provided the neural response is detected after this time window, a neural response measurement can be more easily obtained. This is the case in surgical monitoring where there are large distances (e.g. more than 12 cm for nerves conducting at 60 ms⁻¹) between the stimulus and measurement electrodes so that the propagation time from the stimulus site to the measurement electrodes exceeds 2 ms, which is longer than the typical duration of stimulus artefact.

However, to characterize the responses from the dorsal column, high stimulation currents are required. Similarly, any implanted neuromodulation device will necessarily be of compact size, so that for such devices to monitor the effect of applied stimuli, the stimulus electrode(s) and measurement electrode(s) will necessarily be in close proximity. In such situations the measurement process must overcome artefact directly.

Closed-loop neural stimulation therapy is governed by a number of parameters to which values must be assigned to implement the therapy. The effectiveness of the therapy depends in large measure on the suitability of the assigned parameter values to the patient undergoing the therapy. As patients vary significantly in their physiological characteristics, a “one-size-fits-all” approach to parameter value assignment is likely to result in ineffective therapy for a large proportion of patients. An important preliminary task, once a neuromodulation device has been implanted in a patient, is therefore to assign values to the clinical settings that maximise the effectiveness of the therapy the device will deliver to that particular patient. This task is known as programming or fitting the device. Programming generally involves applying certain test stimuli via the device, recording responses, and based on the recorded responses, inferring or calculating the most effective parameter values for the patient. The resulting parameter values are then formed into a “program” that may be loaded to the device to govern subsequent therapy. Some of the recorded responses may be neural responses evoked by the test stimuli, which provide an objective source of information that may be analysed along with subjective responses elicited from the patient. In an effective programming system, the more responses that are analysed, the more effective the eventual assigned parameter values should be.

However, programming may be costly and time-consuming if unnecessarily prolonged. There is therefore an incentive to minimise the number of test stimuli to be applied and the amount of information to be recorded and analysed in order to produce the assigned values of the clinical settings.

For some patients, it is beneficial for a neural stimulation therapy program to comprise multiple stimulation sets. A stimulation set (“stimset”) is a set of stimulus electrodes along with the stimulus parameters that govern the stimulation pulses delivered via those stimulus electrodes. Each stimset may be independently programmed to target a different painful area, though typically all stimsets have the same stimulus frequency. In such implementations, the stimuli from the multiple stimsets are delivered interleaved in time in a fixed order with a programmable interval between the pulses from each stimset. However, the resources may only be available to analyse the evoked responses from one of the interleaved stimsets, referred to as the applied stimset. In such implementations, the adjustable parameters of the other stimsets may be adjusted based on the evoked responses to the applied stimset. However, when programming multiple interleaved stimsets, it is not apparent which stimset is most suitable to be used as the applied stimset.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood to mean that the element may be any one of the listed options, or may be any combination of two or more of the listed options.

### SUMMARY OF THE INVENTION

Disclosed herein are systems and methods for programming a neuromodulation device with multiple stimulation sets (“stimsets”) to implement closed-loop multi-stimset neural stimulation therapy. The methods and systems according to the disclosed technology assess each stimset in the program to determine a quality metric indicative of the suitability of that stimset to act as the applied stimset for the closed-loop multi-stimset neural stimulation therapy. (The applied stimset is the stimset from whose delivered stimuli the evoked neural responses are measured and used to adjust the parameters for all the stimsets.) The stimset with the highest quality metric is selected as the applied stimset. The applied stimset is then programmed into the neuromodulation device as part of the multiple stimset program to be used in subsequent closed-loop multi-stimset neural stimulation therapy.

According to a first aspect of the present technology, there is provided a neurostimulation system comprising a neurostimulation device for controllably delivering a neural stimulus, and a processor. The neurostimulation device comprises: a plurality of implantable electrodes including one or more stimulus electrodes and one or more sense electrodes; a stimulus source configured to deliver neural stimuli according to a stimulation set to a neural pathway of a patient, wherein the stimulation set comprises a stimulus electrode configuration and a set of stimulus parameters; measurement circuitry configured to capture signal windows from signals sensed at the one or more sense electrodes subsequent to respective neural stimuli; and a control unit configured to control the stimulus source to deliver a neural stimulus according to any one of a plurality of stimulation sets. The processor is configured to: instruct the control unit to control the stimulus source to deliver a plurality of neural stimuli according to a first stimulation set of the plurality of stimulation sets according to respective stimulus intensity parameters; receive a captured signal window corresponding to each delivered neural stimulus; measure a characteristic of an evoked neural response in each captured signal window; and determine a quality metric for the first stimulation set from the measured characteristics.

According to a second aspect of the present technology, there is provided an automated method of controllably delivering neural stimuli. The method comprises: delivering, according to a first stimulation set of a plurality of stimulation sets, the neural stimuli to a neural pathway of a patient according to respective stimulus intensity parameters, wherein each stimulation set comprises a stimulus electrode configuration and a set of stimulus parameters; capturing a signal window subsequent to each delivered neural stimulus; measuring a characteristic of an evoked neural response in each captured signal window; and determining a quality metric for the first stimulation set from the measured characteristics.

According to a third aspect of the present technology, there is provided a neurostimulation system comprising a closed-loop multiple-stimset neurostimulation device, and a processor. The closed-loop multiple-stimset neurostimulation device is configured to controllably deliver neural stimuli according to a plurality of stimulation sets to a neural pathway of a patient so as to maintain a neural response intensity for an applied stimulation set of the plurality of stimulation sets at a corresponding target value. The processor is configured to: instruct the closed-loop multiple-stimset neurostimulation device to deliver a plurality of neural stimuli according to a first stimulation set of the plurality of stimulation sets; receive a captured signal window corresponding to each delivered neural stimulus; measure a characteristic of an evoked neural response in each captured signal window; and determine a quality metric for the first stimulation set from the measured characteristics.

References herein to estimation, determination, comparison and the like are to be understood as referring to an automated process carried out on data by a processor operating to execute a predefined procedure suitable to effect the described estimation, determination and/or comparison step(s). The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be embodied as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored and/or executed in a distributed fashion.

### BRIEF DESCRIPTION OF THE DRAWINGS

One or more implementations of the invention will now be described with reference to the accompanying drawings, in which:

FIG. 1 schematically illustrates an implanted spinal cord stimulator, according to one implementation of the present technology;

FIG. 2 is a block diagram of the stimulator of FIG. 1;

FIG. 3 is a schematic illustrating interaction of the implanted stimulator of FIG. 1 with a nerve;

FIG. 4a illustrates an idealised activation plot for one posture of a patient undergoing neural stimulation;

FIG. 4b illustrates the variation in the activation plots with changing posture of the patient;

FIG. 5 is a schematic illustrating elements and inputs of a closed-loop neural stimulation (CLNS) system, according to one implementation of the present technology;

FIG. 6 illustrates the typical form of an electrically evoked compound action potential (ECAP) of a healthy subject;

FIG. 7 is a block diagram of a neural stimulation therapy system including the implanted stimulator of FIG. 1 according to one implementation of the present technology;

FIG. 8 is an illustration of the stimulus pulses delivered by a stimulation program with four interleaved stimsets;

FIG. 9 is a schematic illustrating elements and inputs of a multi-stimset closed-loop neural stimulation (CLNS) system with multiple stimsets, according to one implementation of the present technology;

FIG. 10 is a flowchart illustrating a method of determining a quality metric for a stimset under test among a plurality of stimsets making up a multi-stimset program, according to one aspect of the present technology; and

FIG. 11 is a flowchart illustrating a method of determining a quality metric for a stimset under test, according to one aspect of the present technology.

### DETAILED DESCRIPTION OF THE PRESENT TECHNOLOGY

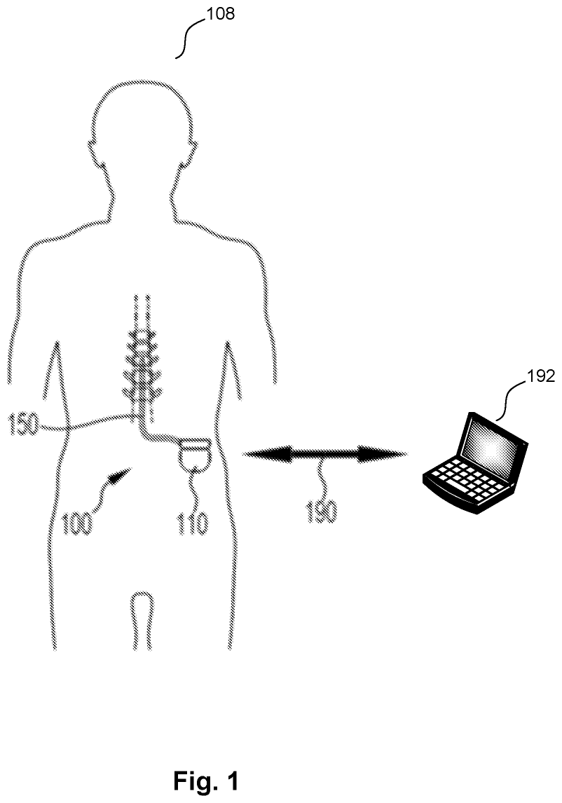

FIG. 1 schematically illustrates an implanted spinal cord stimulator 100 in a patient 108, according to one implementation of the present technology. Stimulator 100 comprises an electronics module 110 implanted at a suitable location. In one implementation, stimulator 100 is implanted in the patient's lower abdominal area or posterior superior gluteal region. In other implementations, the electronics module 110 is implanted in other locations, such as in a flank or sub-clavicularly. Stimulator 100 further comprises an electrode array 150 implanted within the epidural space and connected to the module 110 by a suitable lead. The electrode array 150 may comprise one or more electrodes such as electrode pads on a paddle lead, circular (e.g., ring) electrodes surrounding the body of the lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Numerous aspects of the operation of implanted stimulator 100 may be programmable by an external computing device 192, which may be operable by a user such as a clinician or the patient 108. Moreover, implanted stimulator 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus provide a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface.

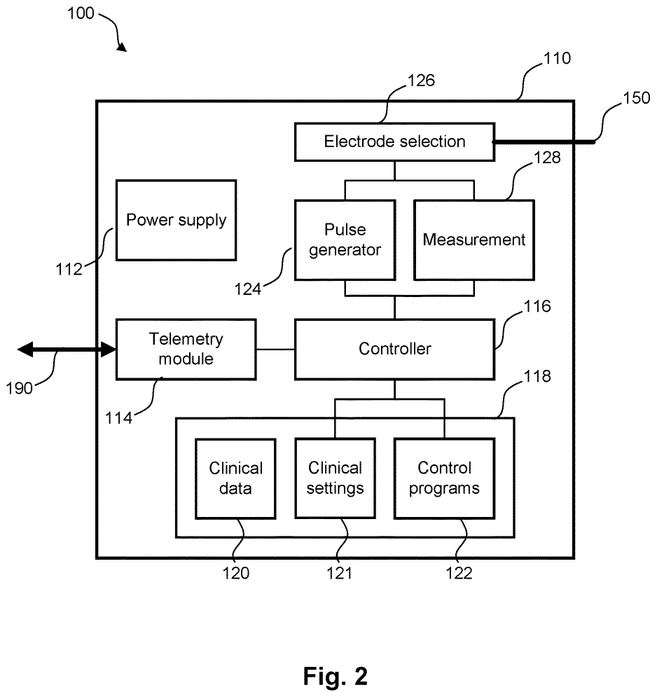

FIG. 2 is a block diagram of the stimulator 100. Electronics module 110 contains a battery 112 and a telemetry module 114. In implementations of the present technology, any suitable type of transcutaneous communications channel 190, such as infrared (IR), radiofrequency (RF), capacitive and/or inductive transfer, may be used by telemetry module 114 to transfer power and/or data to and from the electronics module 110 via communications channel 190. Module controller 116 has an associated memory 118 storing one or more of clinical data 120, clinical settings 121, control programs 122, and the like. Controller 116 is configured by control programs 122, sometimes referred to as firmware, to control a pulse generator 124 to generate stimuli, such as in the form of electrical pulses, in accordance with the clinical settings 121. Electrode selection module 126 switches the generated pulses to the selected electrode(s) of electrode array 150, for delivery of the pulses to the tissue surrounding the selected electrode(s). Measurement circuitry 128, which may comprise an amplifier and/or an analog-to-digital converter (ADC), is configured to process signals comprising neural responses sensed at measurement electrode(s) of the electrode array 150 as selected by electrode selection module 126.

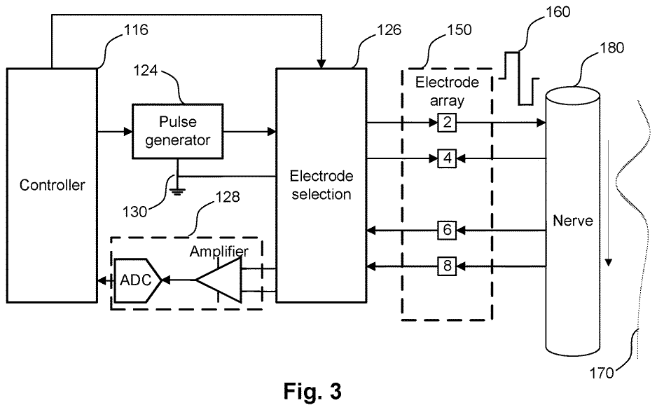

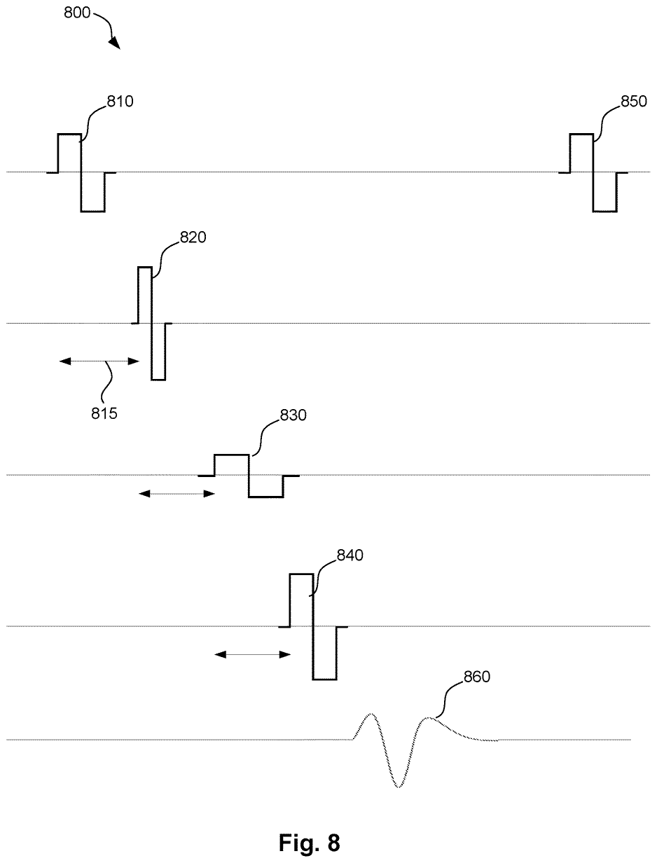

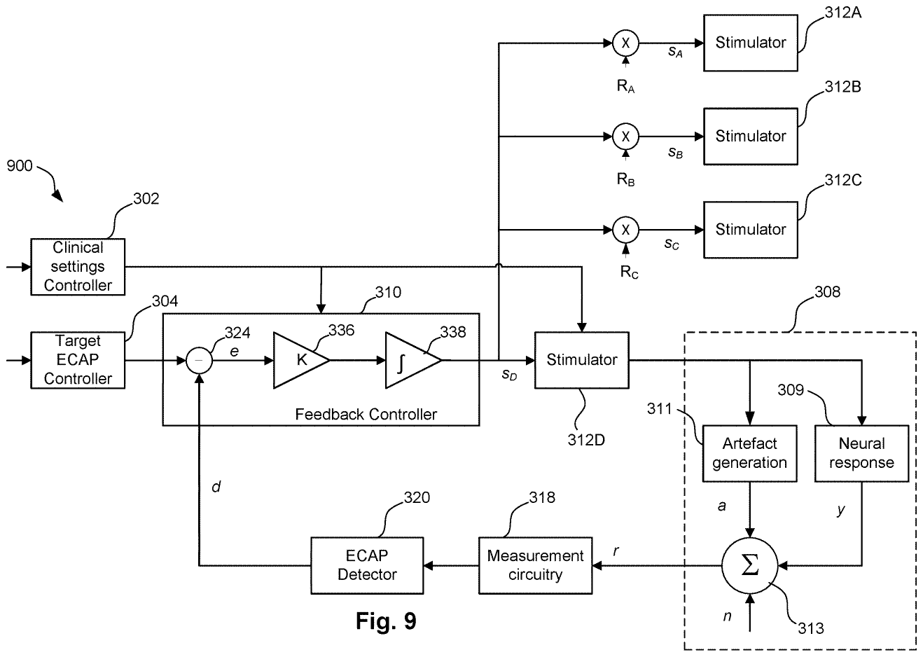

FIG. 3 is a schematic illustrating interaction of the implanted stimulator 100 with a nerve 180 in the patient 108. In the implementation illustrated in FIG. 3 the nerve 180 may be located in the spinal cord, however in alternative implementations the stimulator 100 may be positioned adjacent any desired neural tissue including a peripheral nerve, visceral nerve, parasympathetic nerve or a brain structure. Electrode selection module 126 selects a stimulus electrode 2 of electrode array 150 through which to deliver a pulse from the pulse generator 124 to surrounding tissue including nerve 180. A pulse may comprise one or more phases, e.g. a biphasic stimulus pulse 160 comprises two phases. Electrode selection module 126 also selects a return electrode 4 of the electrode array 150 for stimulus current return in each phase, to maintain a zero net charge transfer. An electrode may act as both a stimulus electrode and a return electrode over a complete multiphasic stimulus pulse. The use of two electrodes in this manner for delivering and returning current in each stimulus phase is referred to as bipolar stimulation. Alternative embodiments may apply other forms of bipolar stimulation, or may use a greater number of stimulus and/or return electrodes. The set of stimulus and return electrodes and their respective polarities is referred to as the stimulus electrode configuration. A stimulation set, as described below in relation to FIGS. 8 and 9, comprises a stimulus electrode configuration (SEC), along with the stimulus parameters that govern the stimulation pulses delivered via that SEC. Electrode selection module 126 is illustrated in FIG. 3 as connecting to a ground 130 of the pulse generator 124 to enable stimulus current return via the return electrode 4. However, other connections for current return may be used in other implementations.

Delivery of an appropriate stimulus via stimulus electrodes 2 and 4 to the nerve 180 evokes a neural response 170 comprising an evoked compound action potential (ECAP) which will propagate along the nerve 180 as illustrated at a rate known as the conduction velocity. The ECAP may be evoked for therapeutic purposes, which in the case of a spinal cord stimulator for chronic pain may be to create paraesthesia at a desired location. To this end, the stimulus electrodes 2 and 4 are used to deliver stimuli periodically at any therapeutically suitable frequency, for example 30 Hz, although other frequencies may be used including frequencies as high as the kHz range. In alternative implementations, stimuli may be delivered in a non-periodic manner such as in bursts, or sporadically, as appropriate for the patient 108. To program the stimulator 100 to the patient 108, a clinician may cause the stimulator 100 to deliver stimuli of various configurations which seek to produce a sensation that is experienced by the user as paraesthesia. When a stimulus electrode configuration is found which evokes paraesthesia in a location and of a size which is congruent with the area of the patient's body affected by pain and of a quality that is comfortable for the patient, the clinician or the patient nominates that configuration for ongoing use. The therapy parameters may be loaded into the memory 118 of the stimulator 100 as the clinical settings 121.

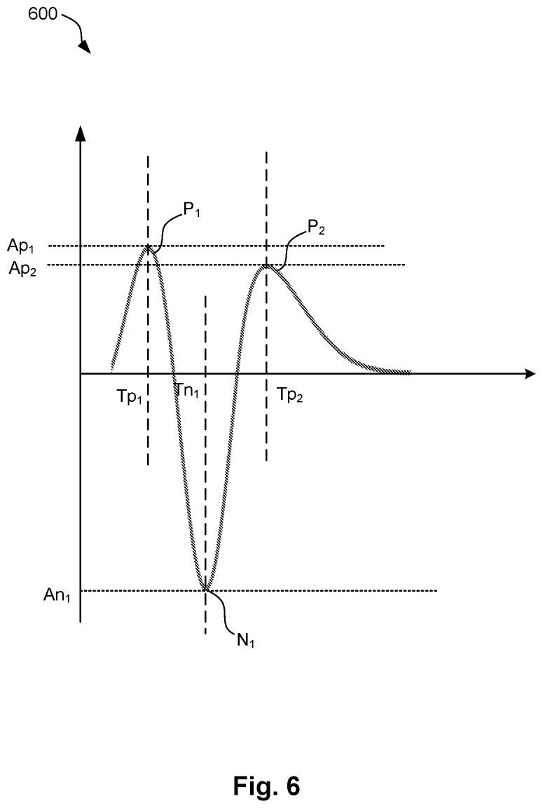

FIG. 6 illustrates the typical form of an ECAP 600 of a healthy subject, as recorded at a single measurement electrode referenced to the system ground 130. The shape and duration of the single-ended ECAP 600 shown in FIG. 6 is predictable because it is a result of the ion currents produced by the ensemble of fibres depolarising and generating action potentials (APs) in response to stimulation. The evoked action potentials (EAPs) generated synchronously among a large number of fibres sum to form the ECAP 600. The ECAP 600 generated from the synchronous depolarisation of a group of similar fibres comprises a positive peak P1, then a negative peak N1, followed by a second positive peak P2. This shape is caused by the region of activation passing the measurement electrode as the action potentials propagate along the individual fibres.

The ECAP may be recorded differentially using two measurement electrodes, as illustrated in FIG. 3. Differential ECAP measurements are less subject to common-mode noise on the surrounding tissue than single-ended ECAP measurements. Depending on the polarity of recording, a differential ECAP may take an inverse form to that shown in FIG. 6, i.e. a form having two negative peaks N1 and N2, and one positive peak P1. Alternatively, depending on the distance between the two measurement electrodes, a differential ECAP may resemble the time derivative of the ECAP 600, or more generally the difference between the ECAP 600 and a time-delayed copy thereof.

The ECAP 600 may be characterised by any suitable characteristic(s) of which some are indicated in FIG. 6. The amplitude of the positive peak P1 is Ap₁ and occurs at time Tp₁. The amplitude of the positive peak P2 is Ap₂ and occurs at time Tp₂. The amplitude of the negative peak P1 is An₁ and occurs at time Tn₁. The peak-to-peak amplitude is Ap1+An₁. A recorded ECAP will typically have a maximum peak-to-peak amplitude in the range of microvolts and a duration of 2 to 3 ms.

The stimulator 100 is further configured to detect the existence and measure the intensity of ECAPs 170 propagating along nerve 180, whether such ECAPs are evoked by the stimulus from electrodes 2 and 4, or otherwise evoked. To this end, any electrodes of the array 150 may be selected by the electrode selection module 126 to serve as recording electrode 6 and reference electrode 8, whereby the electrode selection module 126 selectively connects the chosen electrodes to the inputs of the measurement circuitry 128. Thus, signals sensed by the measurement electrodes 6 and 8 subsequent to the respective stimuli are passed to the measurement circuitry 128, which may comprise a differential amplifier and an analog-to-digital converter (ADC), as illustrated in FIG. 3. The recording electrode and the reference electrode are referred to as the measurement electrode configuration. The measurement circuitry 128 for example may operate in accordance with the teachings of the above-mentioned International Patent Publication No. WO2012/155183.

Signals sensed by the measurement electrodes 6, 8 and processed by measurement circuitry 128 are further processed by an ECAP detector implemented within controller 116, configured by control programs 122, to obtain information regarding the effect of the applied stimulus upon the nerve 180. In some implementations, the sensed signals are processed by the ECAP detector in a manner which measures and stores one or more characteristics from each evoked neural response or group of evoked neural responses contained in the sensed signal. In one such implementation, the characteristics comprise a peak-to-peak ECAP amplitude in microvolts (μV). For example, the sensed signals may be processed by the ECAP detector to determine the peak-to-peak ECAP amplitude in accordance with the teachings of International Patent Publication No. WO2015/074121, the contents of which are incorporated herein by reference. Alternative implementations of the ECAP detector may measure and store an alternative characteristic from the neural response, or may measure and store two or more characteristics from the neural response.

Stimulator 100 applies stimuli over a potentially long period such as days, weeks, or months and during this time may store characteristics of neural responses, clinical settings, paraesthesia target level, and other operational parameters in memory 118. To effect suitable SCS therapy, stimulator 100 may deliver tens, hundreds or even thousands of stimuli per second, for many hours each day. Each neural response or group of responses generates one or more characteristics such as a measure of the intensity of the neural response. Stimulator 100 thus may produce such data at a rate of tens or hundreds of Hz, or even kHz, and over the course of hours or days this process results in large amounts of clinical data 120 which may be stored in the memory 118. Memory 118 is however necessarily of limited capacity and care is thus required to select compact data forms for storage into the memory 118, to ensure that the memory 118 is not exhausted before such time that the data is expected to be retrieved wirelessly by external device 192, which may occur only once or twice a day, or less.

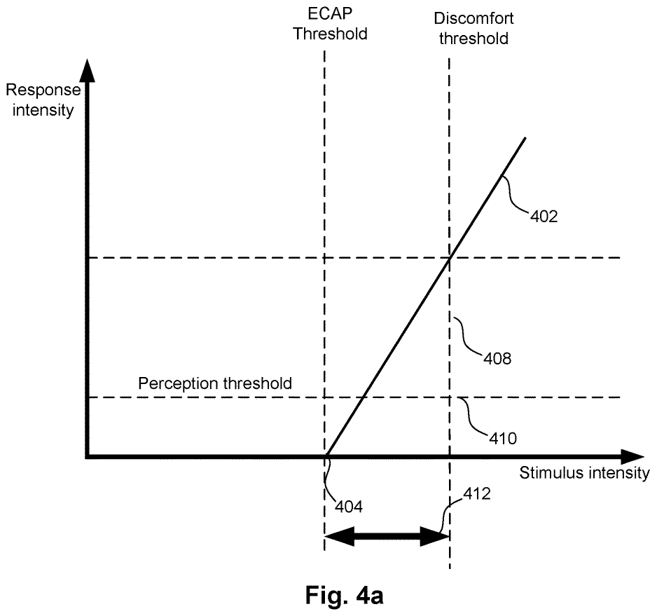

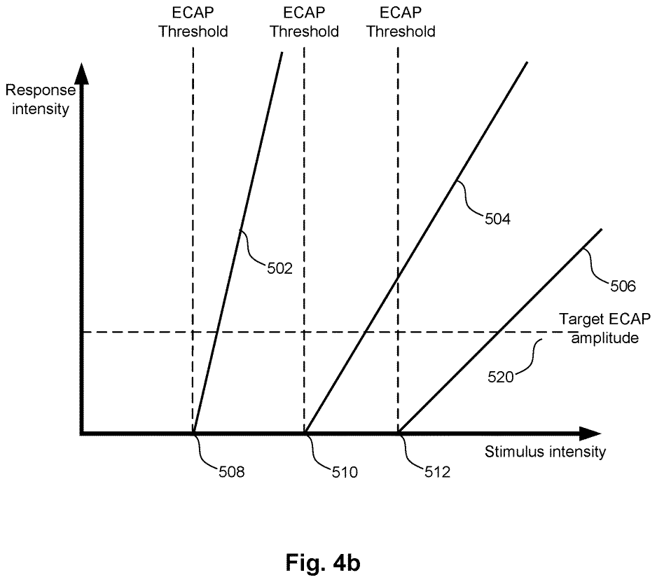

An activation plot, or growth curve, is an approximation to the relationship between stimulus intensity (e.g. an amplitude of the current pulse 160) and intensity of neural response 170 evoked by the stimulus (e.g. an ECAP amplitude). FIG. 4a illustrates an idealised activation plot 402 for one posture of the patient 108. The activation plot 402 shows a linearly increasing ECAP amplitude for stimulus intensity values above a threshold 404 referred to as the ECAP threshold. The ECAP threshold exists because of the binary nature of fibre recruitment; if the field strength is too low, no fibres will be recruited. However, once the field strength exceeds a threshold, fibres begin to be recruited, and their individual evoked action potentials are independent of the strength of the field. The ECAP threshold 404 therefore reflects the field strength at which significant numbers of fibres begin to be recruited, and the increase in response intensity with stimulus intensity above the ECAP threshold reflects increasing numbers of fibres being recruited. Below the ECAP threshold 404, the ECAP amplitude may be taken to be zero. Above the ECAP threshold 404, the activation plot 402 has a positive, approximately constant slope indicating a linear relationship between stimulus intensity and the ECAP amplitude. Such a relationship may be modelled as:

$$y = \{ \begin{matrix}P ( s - T ) , & s \geq T \\ 0 , & s < T\end{matrix} \qquad ( 1 )$$

where s is the stimulus intensity, y is the ECAP amplitude, Tis the ECAP threshold and P is the slope of the activation plot (referred to herein as the patient sensitivity). The slope P and the ECAP threshold T are the key parameters of the activation plot 402.

FIG. 4a also illustrates a discomfort threshold 408, which is a stimulus intensity above which the patient 108 experiences uncomfortable or painful stimulation. FIG. 4a also illustrates a perception threshold 410. The perception threshold 410 corresponds to an ECAP amplitude that is perceivable by the patient. There are a number of factors which can influence the position of the perception threshold 410, including the posture of the patient. Perception threshold 410 may correspond to a stimulus intensity that is greater than the ECAP threshold 404, as illustrated in FIG. 4a, if patient 108 does not perceive low levels of neural activation. Conversely, the perception threshold 410 may correspond to a stimulus intensity that is less than the ECAP threshold 404, if the patient has a high perception sensitivity to lower levels of neural activation than can be detected in an ECAP, or if the signal to noise ratio of the ECAP is low.

For effective and comfortable operation of an implantable neuromodulation device such as the stimulator 100, it is desirable to maintain stimulus intensity within a therapeutic range. A stimulus intensity within a therapeutic range 412 is above the ECAP threshold 404 and below the discomfort threshold 408. In principle, it would be straightforward to measure these limits and ensure that stimulus intensity, which may be closely controlled, always falls within the therapeutic range 412. However, the activation plot, and therefore the therapeutic range 412, varies with the posture of the patient 108.

FIG. 4b illustrates the variation in the activation plots with changing posture of the patient. A change in posture of the patient may cause a change in impedance of the electrode-tissue interface or a change in the distance between electrodes and the neurons. While the activation plots for only three postures, 502, 504 and 506, are shown in FIG. 4b, the activation plot for any given posture can lie between or outside the activation plots shown, on a continuously varying basis depending on posture. Consequently, as the patient's posture changes, the ECAP threshold changes, as indicated by the ECAP thresholds 508, 510, and 512 for the respective activation plots 502, 504, and 506. Additionally, as the patient's posture changes, the slope of the activation plot also changes, as indicated by the varying slopes of activation plots 502, 504, and 506. In general, as the distance between the stimulus electrodes and the spinal cord increases, the ECAP threshold increases and the slope of the activation plot decreases. The activation plots 502, 504, and 506 therefore correspond to increasing distance between stimulus electrodes and spinal cord, and decreasing patient sensitivity.

To keep the applied stimulus intensity within the therapeutic range as patient posture varies, in some implementations an implantable neuromodulation device such as the stimulator 100 may adjust the applied stimulus intensity based on a feedback variable that is determined from one or more measured ECAP characteristics. In one implementation, the device may adjust the stimulus intensity to maintain the measured ECAP amplitude at a target response intensity. For example, the device may calculate an error between a target ECAP amplitude and a measured ECAP amplitude, and adjust the applied stimulus intensity to reduce the error as much as possible, such as by adding the scaled error to the current stimulus intensity. A neuromodulation device that operates by adjusting the applied stimulus intensity based on a measured ECAP characteristic is said to be operating in closed-loop mode and will also be referred to as a closed-loop neural stimulation (CLNS) device. By adjusting the applied stimulus intensity to maintain the measured ECAP amplitude at an appropriate target response intensity, such as a target ECAP amplitude 520 illustrated in FIG. 4b, a CLNS device will generally keep the stimulus intensity within the therapeutic range as patient posture varies.

A CLNS device comprises a stimulator that takes a stimulus intensity value and converts it into a neural stimulus comprising a sequence of electrical pulses according to a predefined stimulation pattern. The stimulation pattern is parametrised by multiple parameters including stimulus amplitude, pulse width, number of phases, order of phases, number of stimulus electrode poles (two for bipolar, three for tripolar etc.), and stimulus rate or frequency. At least one of the stimulus parameters, for example the stimulus amplitude, is controlled by the feedback loop.

In an example CLNS system, a user (e.g. the patient or a clinician) sets a target response intensity, and the CLNS device performs proportional-integral-differential (PID) control. In some implementations, the differential contribution is disregarded and the CLNS device uses a first order integrating feedback loop. The stimulator produces stimulus in accordance with a stimulus intensity parameter, which evokes a neural response in the patient. The intensity of an evoked neural response (e.g. an ECAP) is detected, and its amplitude measured by the CLNS device and compared to the target response intensity.

The measured neural response intensity, and its deviation from the target response intensity, is used by the feedback loop to determine possible adjustments to the stimulus intensity parameter to maintain the neural response at the target intensity. If the target intensity is properly chosen, the patient receives consistently comfortable and therapeutic stimulation through posture changes and other perturbations to the stimulus/response behaviour.

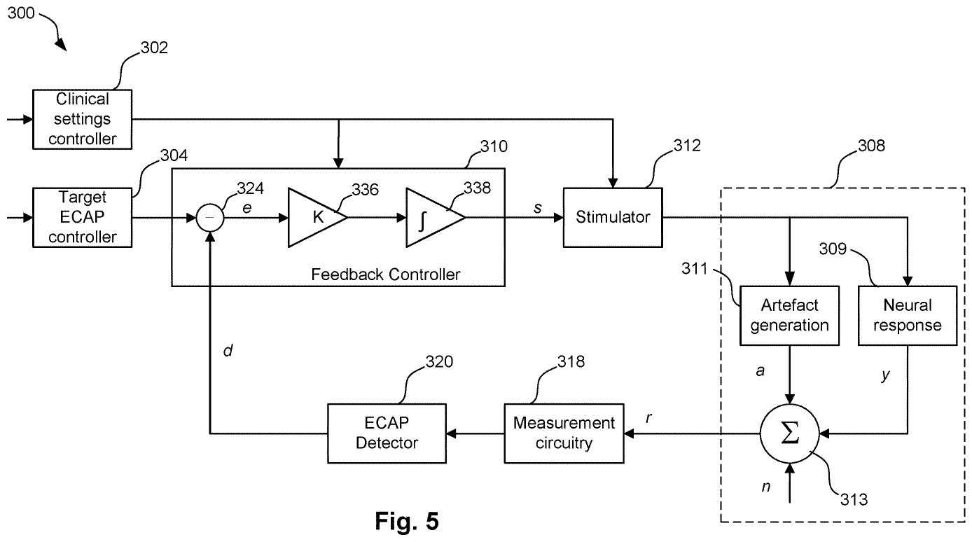

FIG. 5 is a schematic illustrating elements and inputs of a closed-loop neural stimulation (CLNS) system 300, according to one implementation of the present technology. The system 300 comprises a stimulator 312 which converts a stimulus intensity parameter (for example a stimulus current amplitude) s, in accordance with a set of predefined stimulus parameters, to a neural stimulus comprising a sequence of electrical pulses on the stimulus electrodes (not shown in FIG. 5). According to one implementation, the predefined stimulus parameters comprise the number and order of phases, the number of stimulus electrode poles, the pulse width, and the stimulus rate or frequency.

The generated stimulus crosses from the electrodes to the spinal cord, which is represented in FIG. 5 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrodes. Various sources of measurement noise n, as well as the artefact a, may add to the evoked response y at the summing element 313 to form the sensed signal r, including: electrical noise from external sources such as 50 Hz mains power; electrical disturbances produced by the body such as neural responses evoked not by the device but by other causes such as peripheral sensory input; EEG; EMG; and electrical noise from measurement circuitry 318.

The neural recruitment arising from the stimulus is affected by mechanical changes, including posture changes, walking, breathing, heartbeat and so on. Mechanical changes may cause impedance changes, or changes in the location and orientation of the nerve fibres relative to the electrode array(s). As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the voltage resulting from the stimulus applied to evoke the response is typically several volts.

Measurement circuitry 318, which may be identified with measurement circuitry 128, amplifies the sensed signal r (including evoked neural response, artefact, and measurement noise), and samples the amplified sensed signal r to capture a “signal window” comprising a predetermined number of samples of the amplified sensed signal r. The ECAP detector 320 processes the signal window and outputs a measured neural response intensity d. A typical number of samples in a captured signal window is 60. In one implementation, the neural response intensity comprises a peak-to-peak ECAP amplitude. The measured response intensity d is input into the feedback controller 310. The feedback controller 310 comprises a comparator 324 that compares the measured response intensity d (also referred to as the feedback variable) to a target ECAP amplitude as set by the target ECAP controller 304 and provides an indication of the difference between the measured response intensity d and the target ECAP amplitude. This difference is the error value, e.

The feedback controller 310 calculates an adjusted stimulus intensity parameter, s, with the aim of maintaining a measured response intensity d equal to the target ECAP amplitude. Accordingly, the feedback controller 310 adjusts the stimulus intensity parameter s to minimise the error value, e. In one implementation, the controller 310 utilises a first order integrating function, using a gain element 336 and an integrator 338, in order to provide suitable adjustment to the stimulus intensity parameter s. According to such an implementation, the current stimulus intensity parameter s may be computed by the feedback controller 310 as

$$s = \int Kedt \qquad ( 2 )$$

where K is the gain of the gain element 336 (the controller gain). This relation may also be represented as

$$δ s = Ke \qquad ( 3 )$$

where δs is an adjustment to the current stimulus intensity parameter s.

A target ECAP amplitude is input to the feedback controller 310 via the target ECAP controller 304. In one embodiment, the target ECAP controller 304 provides an indication of a specific target ECAP amplitude. In another embodiment, the target ECAP controller 304 provides an indication to increase or to decrease the present target ECAP amplitude. The target ECAP controller 304 may comprise an input into the neuromodulation device, via which the patient or clinician can input a target ECAP amplitude, or indication thereof. The target ECAP controller 304 may comprise memory in which the target ECAP amplitude is stored, and from which the target ECAP amplitude is provided to the feedback controller 310.

A clinical settings controller 302 provides clinical settings to the system 300, including the feedback controller 310 and the stimulus parameters for the stimulator 312 that are not under the control of the feedback controller 310. In one example, the clinical settings controller 302 may be configured to adjust the controller gain K of the feedback controller 310 to adapt the feedback loop to patient sensitivity. The clinical settings controller 302 may comprise an input into the CLNS system 300, via which the patient or clinician can adjust the clinical settings. The clinical settings controller 302 may comprise memory in which the clinical settings are stored, and are provided to components of the system 300.

In some implementations, two clocks (not shown) are used, being a stimulus clock operating at the stimulus frequency (e.g. 60 Hz) and a sample clock for sampling the sensed signal r (for example, operating at a sampling frequency of 10 kHz). As the ECAP detector 320 is linear, only the stimulus clock affects the dynamics of the CLNS system 300. On the next stimulus clock cycle, the stimulator 312 outputs a stimulus in accordance with the adjusted stimulus intensity s. Accordingly, there is a delay of one stimulus clock cycle before the stimulus intensity is updated in light of the error value e.

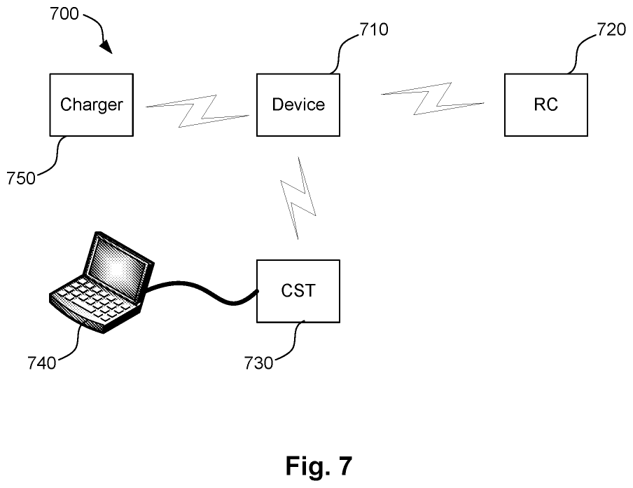

FIG. 7 is a block diagram of a neural stimulation system 700. The neural stimulation system 700 is centred on a neuromodulation device 710. In one example, the neuromodulation device 710 may be implemented as the stimulator 100 of FIG. 1, implanted within a patient (not shown). The neuromodulation device 710 is connected wirelessly to a remote controller (RC) 720. The remote controller 720 is a portable computing device that provides the patient with control of their stimulation in the home environment by allowing control of the functionality of the neuromodulation device 710, including one or more of the following functions: enabling or disabling stimulation; adjustment of stimulus intensity or target neural response intensity; and selection of a stimulation control program from the control programs stored on the neuromodulation device 710.

The charger 750 is configured to recharge a rechargeable power source of the neuromodulation device 710. The recharging is illustrated as wireless in FIG. 7 but may be wired in alternative implementations.

The neuromodulation device 710 is wirelessly connected to a Clinical System Transceiver (CST) 730. The wireless connection may be implemented as the transcutaneous communications channel 190 of FIG. 1. The CST 730 acts as an intermediary between the neuromodulation device 710 and the Clinical Interface (CI) 740, to which the CST 730 is connected. A wired connection is shown in FIG. 7, but in other implementations, the connection between the CST 730 and the CI 740 is wireless.

The CI 740 may be implemented as the external computing device 192 of FIG. 1. The CI 740 is configured to program the neuromodulation device 710 and recover data stored on the neuromodulation device 710. This configuration is achieved by program instructions collectively referred to as the Clinical Programming Application (CPA) and stored in an instruction memory of the CI 740.

### The Assisted Programming System

As mentioned above, obtaining patient feedback about their sensations is important during programming of closed-loop neural stimulation therapy, but mediation by trained clinical engineers is expensive and time-consuming. It would therefore be advantageous if patients could program their own implantable device themselves, or with some assistance from a clinician. However, interfaces for current programming systems are non-intuitive and generally unsuitable for direct use by patients because of their technical nature. There is therefore a need for a CPA to be as intuitive for non-technical users as possible while avoiding discomfort to the patient.

Implementations of an Assisted Programming System (APS) according to the present technology are generally configured to meet this need.

In some implementations, the APS comprises two elements: the Assisted Programming Module (APM), which forms part of the CPA, and the Assisted Programming Firmware (APF), which forms part of the control programs 122 executed by the controller 116 of the electronics module 110. The data obtained from the patient is analysed by the APM to determine the parameters and settings for the neural stimulation therapy to be delivered by the stimulator 100. The APF is configured to complement the operation of the APM by responding to commands issued by the APA via the CST 730 to the stimulator 100 to deliver specified stimuli to the patient, and by returning, via the CST 730, measurements of neural responses to the delivered stimuli.

In other implementations, all the processing of the APS according to the present technology is done by the APF. In other words, the data obtained from the patient is not passed to the APM, but is analysed by the APF to determine the parameters and settings for the neural stimulation therapy to be delivered by the stimulator 100.

In implementations of the APS in which the APM analyses the data from the patient, the APS instructs the device 710 to capture and return signal windows to the CI 740 via the CST 730. In such implementations, the device 710 captures the signal windows using the measurement circuitry 128 and bypasses the ECAP detector 320, storing the data representing the raw signal windows temporarily in memory 118 before transmitting the data representing the captured signal windows to the APS for analysis.

Following the processing, the APS may load the determined program onto the device 710 to govern subsequent neural stimulation therapy. In one implementation, the program comprises clinical settings 121 that are input to the neuromodulation device by, or stored in, the clinical settings controller 302. The patient may subsequently control the device 710 to deliver the therapy according to the determined program using the remote controller 720 as described above. The determined program may also, or alternatively, be loaded into the CPA for validation and modification.

### Multiple Stimset Programming

As mentioned above, for some patients, it is beneficial for a neural stimulation therapy program to comprise multiple stimulation sets. A stimulation set (“stimset”) comprises a set of stimulus and return electrodes, or more precisely a stimulus electrode configuration (SEC), along with the stimulus parameters that govern the stimulation pulses delivered via that SEC.

FIG. 8 is an illustration 800 of the stimulus pulses delivered by a stimulation program with four interleaved stimsets. The stimulus pulse train delivered according to each stimset is illustrated on a separate, but vertically aligned, horizontal axis representing time. All the stimulus pulse trains are delivered at the same stimulus frequency. (It is not a requirement that all the stimulus pulse trains for the respective stimsets are delivered at the same stimulus frequency; however it is so represented in FIG. 8 for ease of illustration.) The first stimulus pulse 810, delivered according to the first stimset, is illustrated as a biphasic, anodic-first stimulus pulse, though many other stimulus pulse types are contemplated. The second, third, and fourth stimulus pulses 820, 830, and 840, delivered according to the second, third, and fourth stimsets in the program respectively, are also biphasic, anodic-first stimulus pulses with different pulse widths and different amplitudes. Each stimulus pulse is illustrated as delayed in time by a constant amount (the inter-stimulus interval, or ISI, 815) from the stimulus pulse delivered according to the preceding stimset. However, this is not to be interpreted as limiting, since the intervals between the pulses in the various stimsets may be different. Because all the stimulus pulse trains in FIG. 8 are delivered at the same stimulus frequency, the four stimulus pulses 810, 820, 830, 840 form a cycle that repeats indefinitely without any change to the relative timing of the pulses from the different stimsets. The fifth stimulus pulse 850 is a subsequent pulse in the pulse train delivered according to the first stimset and is therefore illustrated on the same time axis as the first stimulus pulse 810, and the cycle repeats thereafter.

Also illustrated is an evoked neural response in the form of an evoked compound action potential (ECAP) 860 as sensed through a predetermined measurement electrode configuration (MEC) on a common time axis with the stimulus pulses. The illustrated ECAP 860 is evoked by the fourth stimulus pulse 840. A closed-loop neural stimulation (CLNS) system programmed with multiple interleaved stimsets, as illustrated in FIG. 8, may be based on measurements of the ECAP 860. That is to say, closed-loop adjustments to the stimulus parameters of all stimsets may all be based on measurements of the ECAP 860 from a single stimset, referred to as the applied stimset. In FIG. 8, the final stimset in the cycle is the applied stimset.

If the ISI 815 is short, ECAPs evoked by the first three stimulus pulses 810, 820, and 830 are potentially obscured by stimulus crosstalk and/or artefact from the stimulus pulses 820, 830, and 840. Therefore, if the ISI 815 is short, only the final stimset in the cycle may evoke a measurable ECAP. If the ISI 815 is greater than the refractory period and is sufficiently long that ECAPs evoked by the earlier stimsets are not obscured by stimulus crosstalk and artefact from the other stimulus pulses in the cycle, any of the stimsets in the cycle may evoke a measurable ECAP.

FIG. 9 is a schematic illustrating elements and inputs of a multi-stimset CLNS system 900 with multiple stimsets, according to one implementation of the present technology. The multi-stimset CLNS system 900 is the same as the CLNS system 300 of FIG. 5, with like labels indicating like elements, with the addition of three further stimsets. The four stimsets are labelled A, B, C, and D and are delivered by stimulators 312A, 312B, 312C, and 312D (the latter of which corresponds to the stimulator 312 in the CLNS system 300) according to respective stimulus intensity parameters SA, SB, SC, and SD, and via respective SECs. The pulses delivered by the stimulators 312A, 312B, 312C, and 312D correspond to the stimulus pulses 810, 820, 830, and 840 of FIG. 8. Stimset D, delivered by the stimulator 312D, is delivered last in the cycle and is the applied stimset from which the ECAP is measured. In the implementation of FIG. 8, the stimulus intensity parameter s_{D} for stimset D is scaled by ratios R_{A}, R_{B}, and R_{C} to obtain the stimulus intensity parameters s_{A}, s_{B}, and s_{C} for stimsets A, B, and C respectively. The ratios R_{A}, R_{B}, and R_{C} are fixed at the ratios of the respective stimulus intensities at which the respective stimsets were originally programmed, to the originally programmed stimulus intensity of the applied stimset D. In such an implementation, the stimulus intensity parameters s_{A}, s_{B}, and s_{C} always remain in fixed ratio with the applied stimulus intensity parameter s_{D} and with each other. This is referred to as ratiometric control. So for example, if the originally programmed stimulus intensities were 1 mA, 2 mA, 4 mA, and 6 mA for the four stimsets A, B, C, and D respectively, the ratios R_{A}, R_{B}, and R_{C} are fixed at programming time at ⅙, ⅓, and ⅔ respectively and form part of the clinical settings 121 of the multi-stimset program. If during therapy the feedback controller 310 adjusts the applied stimset intensity parameter s_{D} to 6.6 mA, the stimulus intensity parameters s_{A}, s_{B}, and s_{C} of the non-applied stimsets are automatically adjusted to 1.1 mA, 2.2 mA, and 4.4 mA respectively. The clinical settings controller 302 provides to the stimulators 312A, 312B, 312C, and 312D the stimulus parameters that are not under the control of the feedback controller 310.

If the ISI is very short, the stimsets may interfere with one another, because some fibres recruited by the previous pulse in the cycle may be in the refractory period for a subsequent pulse from the next stimset in the cycle, dependent on the spatial separation of the respective SECs on the array. Patient sensation is therefore dependent on the ordering of the stimsets in the cycle. If sensation is to be preserved, only the final stimset in the cycle may be used as the applied stimset.

Conversely, if the ISI is long enough that such interference is negligible, the order in which the stimsets are delivered is unimportant to patient sensation. In other words, permuting the order in which the stimsets are delivered makes no significant difference to the sensation experienced by the patient. Therefore, any of the stimsets could be last in the cycle and evoke a measurable ECAP. Furthermore, as mentioned above, if the ISI is greater than the refractory period and is sufficiently long that ECAPs evoked by the earlier stimsets are not obscured by stimulus crosstalk and artefact from the other stimulus pulses in the cycle, any or all of the stimsets in the cycle may evoke a measurable ECAP. In such a situation, all stimsets could be independently controlled by their own ECAPs and no single applied stimset need be specified.

However, for the present disclosure, it may be assumed that the ISI is long enough that the order in which the stimsets are delivered is unimportant to patient sensation. In such a situation, any of the stimsets may be placed last in the order to evoke a measurable ECAP. Alternatively, it may be assumed that the ISI is long enough that any or all of the stimsets in the cycle may evoke a measurable ECAP, but there are insufficient processing resources or battery power to measure the characteristics of more than one ECAP in the cycle. Under either assumption, an applied stimset is needed, and there is freedom to choose which stimset is to be the applied stimset.

However, not all of the stimsets are necessarily equally suitable to be the applied stimset. For example, the ECAPs from the applied stimset may be unstable, or the resulting feedback loop may be poor at maintaining the ECAP amplitude of each stimset at the corresponding target ECAP amplitude value through postural perturbations.

Ratiometric control of the non-applied stimsets is effective to maintain each stimset at a constant neural response intensity on the condition that when the patient moves to a new posture, the threshold and slope of all activation plots, both for applied and non-applied stimsets, move in a proportional manner. (See FIG. 4b for examples of activation plots for a given stimset in different postures.)

More explicitly, assuming the relationship between stimulus intensity s and measured response intensity d for any stimset follows Equation (1) (the linear activation plot model), then for stimsets 1 and 2 in their linear regions,

$${d}_{1} ( {s}_{1} ) = {P}_{1} ( {s}_{1} - {T}_{1} ) \qquad ( 4 )$$

$$and$$

$${d}_{2} ( {s}_{2} ) = {P}_{2} ( {s}_{2} - {T}_{2} ) \qquad ( 5 )$$

where Pᵢ is the sensitivity (slope) and Tᵢ is the threshold (intercept) of the respective activation plots. Pᵢ and Tᵢ both in general vary with the electrode-cord distance x which is a proxy for posture.

Now let x change to x′ as the patient moves to a second posture. If P₂ varies with x in the same proportion as P₁, and T₁ and T₂ vary with x in inverse proportion to the variation in P₁, i.e.

$$\frac{{P}_{1} ( x )}{{P}_{1} ( {x}^{′} )} = \frac{{P}_{2} ( x )}{{P}_{2} ( {x}^{′} )} = \frac{{T}_{1} ( {x}^{′} )}{{T}_{1} ( x )} = \frac{{T}_{2} ( {x}^{′} )}{{T}_{2} ( x )} \qquad ( 6 )$$

then it may be shown that keeping s₂ in any fixed ratio with s₁ as x varies keeps d₂ constant as long as d₁ is kept constant. In other words, if Equation (6) holds, a ratiometric multi-stimset CLNS system (e.g. the multi-stimset CLNS system 900) that varies s₁ to keep d₁ constant as posture varies and varies s₂ in fixed ratio with s₁ will also keep d₂ constant as posture varies. Because neural response intensity is a measurable proxy for the therapeutic effect of a stimset, if Equation (6) holds, a ratiometric multi-stimset CLNS system will therefore maintain the therapeutic effect of all stimsets, as long as they all stay within the linear regions of their respective activation plots.

Note that Equation (6) holds if and only if:
P₂ varies with x in the same proportion as P₁, i.e.

$$\frac{{P}_{1} ( x )}{{P}_{1} ( {x}^{′} )} = \frac{{P}_{2} ( x )}{{P}_{2} ( {x}^{′} )} \qquad ( 7 )$$

(the proportionality condition between stimsets 1 and 2) and
P and T for each stimset are in inverse proportion to each other regardless of posture, i.e.

$${P}_{i} ( x ) {T}_{i} ( x ) = {P}_{i} ( {x}^{′} ) {T}_{i} ( {x}^{′} ) = {k}_{i} \qquad ( 8 )$$

where kᵢ is a constant for each stimset i (the inverse proportionality condition of stimset i). Equation (8) is equivalent to the condition that all activation plots across postures for stimset i intersect on the neural response intensity axis at the same point (0,−kᵢ).

Following Equation (6), the proportionality condition in Equation (7) may equivalently be written in terms of thresholds T₁(x), T₁(x′), T₂(x), and T₂(x′).

If Equation (6) does not hold, then varying s₂ in fixed ratio with s₁ will not keep d₂ constant even if d₁ is kept constant for all postures. This means a ratiometric multi-stimset CLNS system will fail to maintain the therapeutic effect of all stimsets for at least some posture changes.

The systems and methods according to the present technology are therefore directed to determining a quality metric for a given stimset in a multi-stimset program. The quality metric is indicative of the suitability of that stimset to act as the applied stimset for a ratiometric multi-stimset CLNS therapy that is to be implemented via the program. The systems and methods according to the present technology are most suitable for multi-stimset CLNS systems in which a choice among the stimsets may be, and needs to be, made as to which stimset will be the applied stimset. For example, the present technology may be useful in a scenario in which the ISI is set to be the longest possible interval, namely the stimulus period divided by the number of stimsets.

In some implementations, the quality metric is determined for all stimsets in the program at programming time, for example by the APS described above. The APS may then select the stimset with the highest quality metric as the applied stimset. The choice of the applied stimset forms part of the multi-stimset program to be downloaded to the device 710 and used in subsequent multi-stimset CLNS therapy.

In other implementations, the device 710 itself may determine the quality metric for the current applied stimset during multi-stimset CLNS therapy. If the quality metric falls below a threshold, an indication may be transmitted to the user that the program needs manual attention. Alternatively, the device 710 may determine the quality metric for all stimsets in the program and, if there is a stimset with a higher quality metric than the current applied stimset, the device 710 may select the stimset with the higher quality metric as the new applied stimset with which to continue the multi-stimset CLNS therapy.

The quality metric is determined by applying test stimuli according to the stimset under test, and possibly according to the other stimsets, and analysing the neural responses (ECAPs) evoked by the test stimuli. The quality metric may be a composite metric made up of one or more individual metrics. Some of the individual metrics relate solely to the stimset under test. Such metrics may be determined independently of the other stimsets, and are therefore referred to as “independent” metrics. Some examples of independent metrics are:
Quality of ECAP measurements across stimulus intensity levels.
Variability of ECAP signal-to-noise ratio or signal-to-artefact ratio at the target ECAP amplitude, or artefact level in the ECAP at low (e.g. sub-threshold) stimulus intensities as posture changes.
Consistency of sensitivity (slope of activation plot) through posture changes.
Inverse proportionality of activation plot parameters through posture changes.
Stability of a feedback loop driven by the stimset under test.

A stimset with unsatisfactory independent metrics is likely to be unsuitable as the applied stimset. However, even a stimset with good independent metrics may be unsuitable as the applied stimset. Other metrics that may play into the overall quality metric therefore relate to the “representativeness” of the stimset under test of the totality of stimsets in the program, i.e. the ability of a feedback loop driven by the stimset under test to maintain constant response intensity across all stimsets through changes in posture. Examples of such “representativeness” metrics are:
Sum or average of RMS noise in neural response intensities across all stimsets in different postures.
Covariance of sensitivity among the multiple stimsets with posture variation. This metric reflects how similar the variation in sensitivity with posture is between the stimset under test and the other stimsets in the program.
Proportionality of sensitivity among the multiple stimsets with posture variation. This metric reflects the similarity of the scaling in sensitivity with posture between the stimset under test and the other stimsets in the program.

An example of a stimset with low representativeness by the second or third metric in the above list is one whose sensitivity falls with a certain posture change while for the other stimsets the sensitivity rises.

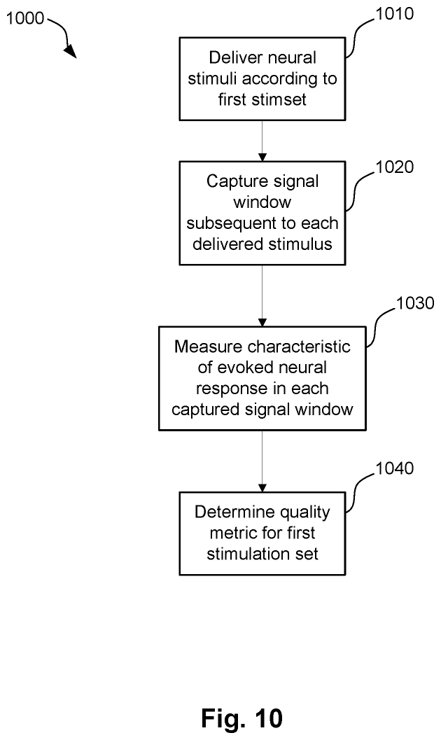

FIG. 10 is a flowchart illustrating a method 1000 of determining a quality metric for a stimset under test among a plurality of stimsets making up a multi-stimset program, according to one aspect of the present technology. The operation of the method 1000 will be described in terms of an APS implementation, but it will be understood that a device-based (e.g. using the APF) implementation is also encompassed by the description.

The method 1000 starts at step 1010, which delivers neural stimuli according to the first stimset. Step 1020 then captures a signal window subsequent to each delivered stimulus as described above. The next step 1030 then measures a characteristic of an evoked neural response in each captured signal window as described above. Finally, at step 1040, the APS determines a quality metric for the first stimset from the measured characteristics.

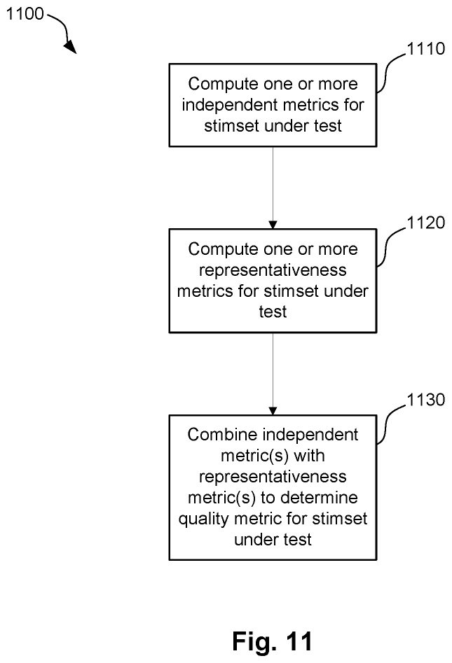

FIG. 11 is a flowchart illustrating a method 1100 of determining a quality metric for a stimset under test. The method 1100 is one implementation of step 1040 of the method 1000, according to an aspect of the present technology.

The method 1100 starts at step 1110, which computes one or more independent metrics for the stimset under test, using measurements of characteristics of evoked responses to stimuli at the stimset under test alone. Methods of computation of various independent metrics to implement step 1110 are described in detail below.

At step 1120, the APS computes one or more representativeness metrics for the stimset under test, using measurements of characteristics of evoked responses to stimuli at all stimsets. Methods of computation of various representativeness metrics to implement step 1120 are described in detail below.

Computation of representativeness metrics as at step 1120 is assisted by the ability to stimulate at, and measure neural responses from, all stimsets near-simultaneously through various postural changes. If this is not practical for a given neuromodulation device, the neural response measurements may be made sequentially at the different stimsets through the same postural change, repeatedly applied.

At step 1130, the APS combines the one or more independent metrics with the one or more representativeness metrics to determine the overall quality metric for the stimset under test.

In the method 1100, either of step 1110 and step 1120 may be omitted, in which case step 1130 merely combines all the individual metrics computed at the non-omitted step of step 1110 and step 1120 into the quality metric.

The individual metrics (independent and/or representativeness) may be combined to determine the overall quality metric at step 1130 of the method 1100 in various ways. In one implementation, each individual metric may be determined on a numeric scale. Each individual metric may be mapped to a uniform scale, such as 0 to 100, and a weighted sum of the individual metrics may be computed to produce a quality metric on the uniform scale. On example of mapping a metric m which can take on any positive value to a value M on the scale of 0 to 100 is:

$$M = 100 \frac{1 - exp ( - m )}{1 + exp ( - m )} \qquad ( 9 )$$

The weightings may be determined empirically based on accumulated clinical data describing what stimsets were selected as the applied stimset in multi-stimset programs and the values of the individual metrics in those programs.

For each stimset, the APS defines at least one measurement electrode configuration (MEC) through which to make measurements of characteristics of evoked responses. In one implementation, an MEC for a tripolar stimset comprises a recording electrode separated by four contacts from the central electrode of the tripole, and a reference electrode separated by a further two electrodes from the recording electrode. However, other choices for an MEC for a given stimset are possible.

Turning to the computation of independent metrics for a stimset under test, as in step 1110 of the method 1100, there are two classes of independent metrics: those requiring measurements of characteristics of evoked responses across multiple stimulus intensities (cross-intensity metrics), and those requiring measurements of characteristics of evoked responses across multiple postures (cross-posture metrics).

One example of an independent cross-intensity metric is an activation plot quality metric. Multiple stimuli are delivered through the stimset under test at intensities spanning the therapeutic range, and intensities of the evoked responses are measured using an ECAP detector. To estimate the therapeutic range for a stimset, the ECAP threshold T may first be estimated using prior patient data comprising ECAP thresholds for many patients, together with their characteristics. In one such implementation, ECAP thresholds from patients with similar characteristics to the current patient 108, for example the absolute position of the stimset in relation to the spinal cord, are retrieved from the patient data, and a representative ECAP threshold value is extracted from the retrieved ECAP thresholds. The APS may then infer a discomfort threshold Max at the stimset from the ECAP threshold T at that stimset. In one implementation, the APS uses a linear prediction model:

$$Max = m \cdot T \qquad ( 10 )$$

where m is a correlation parameter that may be derived from patient data comprising many values of ECAP threshold T and corresponding values of discomfort threshold Max at a given stimset. In one implementation, m takes a value between 1.0 and 2.0. In another implementation, m takes a value between 1.1 and 1.6. In one implementation, m takes a value between 1.25 and 1.5.

To obtain the activation plot quality metric, the APS instructs the device 710 to deliver stimuli of varying intensities Iᵢ between the ECAP threshold T and the discomfort threshold Max according to the stimset under test and to return the corresponding captured signal windows. The APS then uses an ECAP detector to measure a response intensity Eᵢ for each captured signal window. The APS thus forms a set of stimulus intensity-response intensity pairs {(Iᵢ, Eᵢ), I=1, . . . , N}, where N>1, for the stimset. The APS then uses the set of pairs {(Iᵢ, Eᵢ), i=1, . . . , N} for each SEC to estimate the activation plot quality for the stimset under test.

In many embodiments, the ECAP detector is configured to account for the ECAP shape and duration resulting from the offset of the MEC from the stimset under test. Configuration of ECAP detectors is described in the above-mentioned International Patent Publication No. WO2015/074121.

In one implementation of determining an activation plot quality metric, a straight line is fit to the pairs (Iᵢ, Eᵢ), for example using conventional linear regression. As modelled by Equation (1), the slope and x-intercept of the fitted line are the sensitivity P and ECAP threshold T for the stimset under test. The APS may determine the activation plot quality metric by dividing the size of the therapeutic range (Max−T) by the standard deviation of the residuals of the fitted line.

In an alternative implementation of determining an activation plot quality metric, the APS may fit a model referred to as the Logistic Growth Curve (LGC) to the pairs (Iᵢ, Eᵢ) for the stimset under test. In one implementation, the LGC model is a four-parameter function of stimulus intensity I:

$$E ( I ) = A + \frac{K - A}{1 + exp ( - B ( I - M ) )} \qquad ( 11 )$$

where the four parameters are:
A, the minimum value (the detected ECAP amplitude in the absence of stimulation)
K, the maximum value (the detected ECAP amplitude at which saturation occurs, i.e. increases in stimulus intensity do not increase the detected ECAP amplitude)
M, the current amplitude at the midpoint between A and K.
B, the steepness of the LGC, which is proportional to the gradient at the midpoint between A and K.

To fit the LGC, the parameters A, K, M, and B may be initialised to sensible starting points A₀, K₀, M₀, and B₀. In one implementation, these values may be set to:
A₀: the mean of the ECAP amplitudes obtained from the lowest few stimulus current amplitudes.
K₀: the mean of the ECAP amplitudes obtained from the highest few stimulus current amplitudes.
M₀: the stimulus current amplitude at the midpoint between A and K.
B₀: may be calculated from the gradient m at the midpoint, obtained from local linear regression of pairs (Iᵢ, Eᵢ) acquired near the midpoint, as B₀=m*4/(K₀−A₀).

An optimisation algorithm such as Trust Region Reflective (TRF) may then be used to optimise the four parameters A, K, M, and B from their starting points A₀, K₀, M₀, and B₀.

The fitted LGC may be used to estimate the ECAP threshold T at the stimset under test. In one implementation, a line is constructed through the midpoint M of the fitted LGC with slope B. The ECAP threshold T may be estimated as the stimulus current amplitude s at which the constructed line intersects the minimum value A. It may be shown that the resulting ECAP threshold T is given by

$$T = M - \frac{2}{B} \qquad ( 12 )$$

The fitted LGC may also be used to estimate the patient sensitivity P at the stimset under test. In one implementation, the patient sensitivity P is the slope of the fitted LGC at its midpoint M, which may be computed from the steepness B as follows:

$$S = \frac{B}{4} ( K - A ) \qquad ( 13 )$$

The APS may also determine the activation plot quality metric as the growth curve quality index (GCQI) for the fitted LGC model. The GCQI indicates a signal-to-noise ratio (SNR) of the fitted LGC. In one implementation, the APS may calculate the GCQI by dividing the peak-to-peak amplitude of the fitted LGC (K--A) by the standard deviation of the residuals of the fitted LGC.

To obtain independent metrics requiring measurements of characteristics of responses to stimuli across multiple postures (cross-posture metrics), the patient is first instructed to assume a candidate posture. The candidate posture is one of standing, sitting, lying down, prone, lying on side with back straight, lying on side with back arched, and any other posture that the patient might commonly find themselves in. For measurements of characteristics involving ECAPs, stimulus intensity may be set to a comfortable and therapeutic level within the therapeutic range for that posture and the stimset under test. One example of a comfortable level is the stimulus intensity corresponding to the target ECAP amplitude in each posture. Alternatively, for artefact-only characteristics, stimulus intensity may be set to a sub-threshold level, which has the advantage of not being perceptible by the patient. The APS instructs the device 710 to deliver a stimulus according to the stimset under test at the chosen stimulus intensity and capture the subsequent signal window. A source separation algorithm is applied to isolate any ECAP component and any artefact components from the captured signal window. International Patent Publication no. WO2020/124135 by the present applicant, the contents of which are herein incorporated by reference, discloses a source separation method that may be applied to the captured signal window in one implementation.

Morphological ECAP features, a signal-to-noise ratio (SNR), an artefact level, and a signal-to-artefact ratio (SAR) may be calculated as described below from the isolated components. If signal windows have not been captured from all candidate postures, then the patient is placed in the next candidate posture and the above-described routine is performed again. Once all candidate postures have been traversed, the morphological ECAP features, SNRs, artefact level, and SARs acquired from the different candidate postures are used to calculate one or more cross-posture metrics for the stimset under test.

Morphological features of the isolated ECAP component may comprise one or more of: a position or a width of an ECAP peak such as the P1, N1, or P2 peaks; or a maximum slope between adjacent ECAP peaks, such as between the P1 and N1 peaks, or between the N1 and P2 peaks.

High SNR and SAR are indicative of better control of neural recruitment. Specifically, SAR stability across multiple postures is often desirable for more precise recruitment control. SNR, artefact level, and SAR may be calculated in similar ways using the ECAP and artefact components obtained via source separation of the signal window. In one implementation, SNR is calculated by subtracting the artefact and ECAP components from the signal window to obtain a residual (noise) signal, and subsequently calculating SNR as:

$$S N R = \frac{{V}_{rms} ( ECAP )}{{V}_{rms} ( residual )}$$

where Vᵣₘₛ(ECAP) is the root mean square (RMS) value of the ECAP component and Vᵣₘₛ(residual) is the RMS value of the residual. Similarly, the SAR can be calculated as:

$$S A R = \frac{{V}_{rms} ( ECAP )}{{V}_{rms} ( artefact )}$$

where Vᵣₘₛ(artefact) (the artefact level) is the RMS value of the artefact component.

One example of an independent cross-posture metric is the morphological stability, which may be computed as the coefficient of variation of the measurements of a morphological feature across postures. The coefficient of variation of a measurement is a statistical measure of the relative dispersion of the measurements around the mean, and may be computed as the standard deviation of the measurement divided by the mean of the measurement.

Another example of an independent cross-posture metric may be computed as the coefficient of variation of the SNR measurements across the set of postures tested.

Another example of an independent cross-posture metric may be computed as the coefficient of variation of the SAR measurements across the set of postures tested.

Another example of an independent cross-posture metric may be computed as the coefficient of variation of the artefact level measurements across the set of postures tested.

In another implementation of computing an independent cross-posture metric for the stimset under test, the coefficient of variation of patient sensitivity at the stimset under test across postures is computed. As illustrated in FIG. 4b, patient sensitivity can change with posture, as the electrodes get closer to, or further from, the spinal cord. In some implementations, CLNS therapy works better (that is, the loop is more stable) for patients that show less variation in sensitivity with posture than patients who exhibit more variation in sensitivity with posture.

One approach to computing the coefficient of variation of patient sensitivity at the stimset under test across postures is to estimate the sensitivity in each posture by fitting an activation plot to neural response intensity measurements in each posture, and estimating the sensitivity as the slope of the activation plot. The coefficient of variation of the sensitivity across postures may then be computed.

In a CLNS system such as the system 300, when keeping the controller gain K constant, the noise on the measured neural response intensity d, i.e. the feedback variable (FBV), increases monotonically with patient sensitivity P in accordance with the following equation:

$$R = \sqrt{\frac{2}{2 - K P}} \qquad ( 14 )$$

where R is the ratio of the standard deviation of the noise on intensity d in closed-loop mode to the standard deviation of the noise on intensity d in open-loop mode.

Change in sensitivity with posture may therefore be quantified by setting a target for the FBV, closing the loop with the stimset under test to maintain the average FBV at the target, and measuring the standard deviation of the FBV in different postures. The cross-posture sensitivity variation metric may be computed as the coefficient of variation of the standard deviation of the FBV across the different postures. This approach is simpler than generating multiple activation plots across multiple postures, because there is no need to adjust the stimulus intensity and therefore risk over-stimulating the patient during testing. Such an implementation is purely an independent cross-posture metric.

In another implementation of computing an independent cross-posture metric for the stimset under test, sensitivity P and ECAP threshold T may be measured as described above for each posture, and their product k=PT may be computed for each posture. The coefficient of variation of the product k may be computed across the set of postures tested. This metric is a measure of the how closely the stimset under test satisfies the inverse proportionality condition of Equation (8).

Turning now to the computation of representativeness metrics for a stimset under test, as in step 1120 of the method 1100, one implementation comprises setting a target for the FBV, closing the loop with the stimset under test to maintain the average FBV from the stimset under test at the target, and measuring the amount of noise in the neural response intensity evoked by the stimulus pulses from each stimset. This produces a vector of noise amounts (e.g, RMS values or standard deviations) across the stimsets for a given posture. The noise amounts may be combined in some manner, e.g. averaged or summed, into a single value representative of the noise across all stimsets in the given posture. This single value may be repeatedly measured for multiple postures and the measurements combined, e.g. summed or averaged, over all postures to obtain a cross-posture noise value. This cross-posture noise value is representative of the noise across all stimsets and all postures tested. The cross-posture noise value becomes smaller as the representativeness of the stimset under test increases and may therefore be inverted or reciprocated to become a representativeness metric that increases with the representativeness of the stimset under test.

Another implementation of computing a representativeness metric comprises setting a target for the FBV, closing the loop with the stimset under test to maintain the average FBV from the stimset under test at the target, and measuring the amount of noise in the response intensity at each stimset. This produces a vector n of noise amounts (e.g, standard deviations) across the stimsets for a given posture. It will be recalled that in a CLNS system, noise in the measured response intensity from a given stimset is related to (i.e. increases monotonically with) the sensitivity of the patient to stimulation at that given stimset. The amount of noise in the measured response intensity at a stimset may therefore be treated as a proxy for sensitivity at that stimset.

This measurement of a vector n of noise amounts across the stimsets may be repeated for multiple postures. The resulting vectors n₁, . . . , nₚ may be stacked into a noise matrix N that has p rows and n columns, where p is the number of postures tested and n is the number of stimsets in the multi-stimset program. The mean value of each row of the noise matrix N may be subtracted from that row to ensure each row of N has a mean of zero. An n-by-n noise covariance matrix C may then be computed by pre-multiplying the noise matrix N by its transpose:

$${C}_{n} = {N}^{T} N \qquad ( 15 )$$

Each row or column of the noise covariance matrix Cₙ represents the similarity of the variation of the noise amount (and therefore the sensitivity) across postures between a corresponding stimset and the other stimsets. The entries of Cₙ corresponding to the stimset under test (i.e. the entries in the row or column of Cₙ corresponding to the stimset under test) may be combined (e.g. summed or averaged) to obtain the representativeness metric. A high value of this representativeness metric reflects a similarity in the direction and extent of variation of sensitivity across postures between the stimset under test and the ensemble of the other stimsets. This metric therefore indicates suitability of the stimset under test to act as the applied stimset for all the others in a ratiometric multi-stimset CLNS system such as illustrated in FIG. 9.

In an alternative implementation, the sensitivity Pᵢⱼ at each stimset i and each posture j may be directly measured by fitting an activation plot to multiple measurements of neural response intensity across the therapeutic range at that stimset i and posture j as described above. The resulting measurements of sensitivity across n stimsets and p postures may be arranged into a p-by-n sensitivity matrix P. The computation of the representativeness metric in this implementation may then proceed as described above using the sensitivity matrix P rather than the noise matrix N to produce an n-by-n sensitivity covariance matrix C_{P}:

$${C}_{P} = {P}^{T} P \qquad ( 16 )$$

Each row or column of the sensitivity covariance matrix C_{P} represents the similarity of the variation of the sensitivity across postures between a corresponding stimset and the other stimsets. The entries of C_{P} corresponding to the stimset under test (i.e. the entries in the row or column of C_{P} corresponding to the stimset under test) may be combined (e.g. summed or averaged) to obtain the representativeness metric. A high value of this representativeness metric reflects a similarity in the direction and extent of variation of sensitivity P across postures between the stimset under test and the ensemble of the other stimsets. This metric therefore indicates suitability of the stimset under test to act as the applied stimset for all the others in a ratiometric multi-stimset CLNS system such as illustrated in FIG. 9.

In a further alternative implementation, the measurement of sensitivity Pᵢⱼ in stimset i and posture j may be divided by the measurement of sensitivity Pᵢ₁ for stimset i in posture 1 (an arbitrarily chosen reference posture) to form a ratio rᵢⱼ. The ratios rᵢⱼ for i=1 to n and for j=1 to p−1 may be formed into an n-by-(p−1) sensitivity ratio matrix R_{P}. Each row of R_{P} corresponds to a stimset and each column to a posture other than the reference posture.

As mentioned above, the proportionality condition of Equation (7) is part of the conditions in Equation (6) for stimset 1 being suitable to maintain the therapeutic effect of stimset 2 in a ratiometric multi-stimset CLNS system. The ratios rᵢⱼ in the sensitivity ratio matrix R_{P} capture the sensitivity ratios in Equation (6) across all stimsets (as i varies down the rows) and postures (as j varies across the columns). It follows that a stimset i is suitable to act as an applied stimset if the ratio rᵢⱼ in column j (corresponding to posture j+1) of row i is generally equal or close to equal to the other ratios rᵢⱼ in column j, for all columns j.

A representativeness metric Rᵢ according to this implementation may therefore be constructed for a stimset under test (stimset i) by:
computing a median, average, or other representative value r of the ratios r̅ᵢⱼ across all stimsets i in column j;
computing the absolute difference dᵢⱼ between the representative value r̅ⱼ and the ratio rᵢⱼ;
combining (e.g. summing or averaging) the absolute, or squared, differences dᵢⱼ across all postures j (columns) for the row corresponding to the stimset under test (row i) to form the representativeness metric Rᵢ.

In the ideal case of Equation (7) being satisfied across all postures and stimsets, the representativeness metric Rᵢ is zero for the stimset i. A higher value of Rᵢ indicates increasing unsuitability of stimset i to act as the applied stimset for all the others in a ratiometric multi-stimset CLNS system such as illustrated in FIG. 9. The representativeness metric Rᵢ according to this implementation may therefore need to be inverted after being mapped to a uniform scale as in Equation (9) and before being combined with the other metrics in step 1130.

In a further alternative implementation, the above procedure may be carried out on threshold rather than sensitivities to form a threshold ratio matrix R_{T} and compute from R_{T} the representativeness metric Rᵢ. This is because, as mentioned above, the proportionality condition in Equation (7) may equivalently be written in terms of thresholds T₁(x), T₁(x′), T₂(x), and T₂(x′).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not limiting or restrictive.

1. A neurostimulation system comprising:
a neurostimulation device for controllably delivering a neural stimulus, the neurostimulation device comprising:
a plurality of implantable electrodes including one or more stimulus electrodes and one or more measurement electrodes;
a stimulus source configured to deliver neural stimuli according to a stimulation set to a neural pathway of a patient, wherein the stimulation set comprises a stimulus electrode configuration and a set of stimulus parameters;
measurement circuitry configured to capture signal windows from signals sensed at the one or more measurement electrodes subsequent to respective neural stimuli; and
a control unit configured to control the stimulus source to deliver a neural stimulus according to any one of a plurality of stimulation sets; and
a processor configured to:
instruct the control unit to control the stimulus source to deliver a plurality of neural stimuli according to a first stimulation set of the plurality of stimulation sets and according to respective stimulus intensity parameters;
receive from the measurement circuitry a captured signal window subsequent to each delivered neural stimulus;
measure a characteristic of an evoked neural response in each captured signal window; and
determine a quality metric for the first stimulation set from the measured characteristics.

2. The neurostimulation system of claim 1, wherein the processor is configured to determine the quality metric for the first stimulation set from the measured characteristics by computing an independent metric for the first stimulation set.

3. The neurostimulation system of claim 2, wherein the independent metric comprises a cross-intensity metric.

4. The neurostimulation system of claim 3, wherein the processor is configured to compute the cross-intensity metric by:
measuring an intensity of an evoked neural response in each captured signal window, thereby forming a set of stimulus intensity parameter-response intensity pairs;
fitting an activation plot to the set of stimulus intensity parameter-response intensity pairs; and
computing the cross-intensity metric as a quality metric of the fitted activation plot.

5. The neurostimulation system of claim 2, wherein the independent metric comprises a cross-posture metric.

6. The neurostimulation system of claim 5, wherein the processor is configured to compute the cross-posture metric from the measured characteristics of evoked neural responses in captured signal windows corresponding to neural stimuli delivered with the patient in a plurality of postures.

7. The neurostimulation system of claim 6, wherein the processor is configured to compute the cross-posture metric by computing a coefficient of variation of the measured characteristic across the plurality of postures.

8. The neurostimulation system of claim 6, wherein the measured characteristic is a signal-to-noise ratio of the captured signal window.

9. The neurostimulation system of claim 6, wherein the measured characteristic is a signal-to-artefact ratio of the captured signal window.

10. The neurostimulation system of claim 6, wherein the measured characteristic is an artefact level in the captured signal window.

11. The neurostimulation system of claim 6, wherein the measured characteristic is morphological feature of an evoked neural response in the captured signal window.

12. The neurostimulation system of claim 6, wherein the processor is configured to compute the cross-posture metric by:
measuring an intensity of an evoked neural response in each captured signal window, thereby forming a set of stimulus intensity parameter-response intensity pairs;
fitting an activation plot to the set of stimulus intensity parameter-response intensity pairs; and
estimating a sensitivity of the fitted activation plot for a posture of the plurality of postures.

13. The neurostimulation system of claim 12, wherein the processor is configured to compute the cross-posture metric by computing a coefficient of variation of the estimated sensitivities across the plurality of postures.

14. The neurostimulation system of claim 12, wherein the processor is further configured to estimate a threshold of the fitted activation plot for a posture of the plurality of postures.

15. The neurostimulation system of claim 14, wherein the processor is configured to compute the cross-posture metric by computing a coefficient of variation of products of the estimated sensitivities and the estimated thresholds across the plurality of postures.

16. The neurostimulation system of claim 1, wherein the processor is configured to determine the quality metric for the first stimulation set from the measured characteristics by computing a representativeness metric for the first stimulation set.

17. The neurostimulation system of claim 16, wherein the control unit is further configured to:
control the stimulus source to deliver a neural stimulus according to each of the plurality of stimulation sets in a cycle according to respective stimulus intensity parameters;
measure an intensity of an evoked neural response in each captured signal window corresponding to a stimulus delivered according to the first stimulation set;
adjust the stimulus intensity parameter for the first stimulation set based on the measured neural response intensity so as to maintain the measured neural response intensity at a target value; and
adjust the stimulus intensity parameters for the other stimulation sets based on the stimulus intensity parameter for the first stimulation set.

18. The neurostimulation system of claim 17, wherein the processor is configured to compute the representativeness metric for the first stimulation set by:
measuring, for each stimulation set of the plurality of stimulation sets, an intensity of an evoked neural response in each captured signal window corresponding to a stimulus delivered according to the stimulation set; and
computing, for each stimulation set of the plurality of stimulation sets, an amount of noise in the measured neural response intensities corresponding to the stimulation set.

19. The neurostimulation system of claim 18, wherein the processor is further configured to compute the representativeness metric for the first stimulation set by:
combining the amounts of noise into a single measure of noise across all stimulation sets.

20. The neurostimulation system of claim 19, wherein the processor is further configured to compute the representativeness metric for the first stimulation set from the single measure of noise.

21. The neurostimulation system of claim 19, wherein the processor is further configured to compute the representativeness metric for the first stimulation set by:
repeating the measuring, computing, and combining with the patient in at least one further posture to obtain a single measure of noise across all stimulation sets for each posture;
combining the single measures of noise for each posture into a single cross-posture measure of noise across all stimulation sets; and
computing the representativeness metric from the single cross-posture measure of noise.

22. The neurostimulation system of claim 18, wherein the processor is further configured to compute the representativeness metric for the first stimulation set by:
repeating the measuring and computing with the patient in at least one further posture to obtain a single measure of noise across all stimulation sets for each posture;
constructing a noise matrix from the amounts of noise for each stimulation set and each posture;
computing a noise covariance matrix from the noise matrix; and
computing the representativeness metric from a row of entries of the noise covariance matrix corresponding to the first stimulation set.

23. The neurostimulation system of claim 17, wherein the processor is configured to compute the representativeness metric for the first stimulation set by:
measuring, for each stimulation set of the plurality of stimulation sets, a plurality of response intensities of a plurality of evoked neural responses corresponding to respective stimuli delivered according to the stimulation set;
computing, for each stimulation set of the plurality of stimulation sets, a sensitivity corresponding to the stimulation set from the plurality of measured response intensities; and
compute the representativeness metric from the computed sensitivities.

24. The neurostimulation system of claim 23, wherein the processor is configured to compute the representativeness metric as a measure of how closely the sensitivity corresponding the first stimulation set satisfies a proportionality condition with the sensitivities corresponding to the other stimsets.

25. The neurostimulation system of claim 1, wherein the control unit is further configured to:
control the stimulus source to deliver a neural stimulus according to each of the plurality of stimulation sets in a cycle according to respective stimulus intensity parameters;
measure an intensity of an evoked neural response in each captured signal window corresponding to a stimulus delivered according to an applied stimulation set of the plurality of stimulation sets;
adjust the stimulus intensity parameter for the applied stimulation set based on the measured neural response intensity so as to maintain the measured neural response intensity at a target value; and
adjust the stimulus intensity parameters for the non-applied stimulation sets based on the stimulus intensity parameter for the applied stimulation set.

26. The neurostimulation system of claim 25, wherein the quality metric of the first stimulation set is indicative of the suitability of the first stimulation set to be used as the applied stimulation set.

27. The neurostimulation system of claim 26, wherein the processor is further configured to repeat the instructing, receiving, measuring, and determining for each stimulation set of the plurality of stimulation sets, to thereby obtain a quality metric for each stimulation set of the plurality of stimulation sets.

28. The neurostimulation system of claim 27, wherein the processor is further configured to select a stimulation set to be used as the applied stimulation set based on the determined quality metrics for each stimulation set.

29. The neurostimulation system of claim 28, wherein the processor is further configured to program the neurostimulation device to use the selected stimulation set as the applied stimulation set.

30. The neurostimulation system of claim 1, wherein the processor is part of the neurostimulation device.

31. The neurostimulation system of claim 1, further comprising an external computing device in communication with the neurostimulation device.

32. The neurostimulation system of claim 31, wherein the processor is part of the external computing device.

33. An automated method of controllably delivering neural stimuli, the method comprising:
delivering, according to a first stimulation set of a plurality of stimulation sets, the neural stimuli to a neural pathway of a patient according to respective stimulus intensity parameters, wherein each stimulation set comprises a stimulus electrode configuration and a set of stimulus parameters;
capturing a signal window subsequent to each delivered neural stimulus;
measuring a characteristic of an evoked neural response in each captured signal window; and
determining a quality metric for the first stimulation set from the measured characteristics.

34. The method of claim 33, wherein the determining the quality metric for the first stimulation set from the measured characteristics comprises computing an independent metric for the first stimulation set.

35. The method of claim 33, wherein the determining the quality metric for the first stimulation set from the measured characteristics comprises computing a representativeness metric for the first stimulation set.

36. The method of claim 33, further comprising:
delivering a neural stimulus according to each of the plurality of stimulation sets in a cycle according to respective stimulus intensity parameters;
measure an intensity of an evoked neural response in each captured signal window corresponding to a stimulus delivered according to an applied stimulation set of the plurality of stimulation sets;
adjust a stimulus intensity parameter for the applied stimulation set based on the measured neural response intensity so as to maintain the measured neural response intensity at a target value; and
adjust the stimulus intensity parameters for the non-applied stimulation sets based on the stimulus intensity parameter for the applied stimulation set.

37. The method of claim 36, wherein the quality metric of the first stimulation set is indicative of the suitability of the first stimulation set to be used as the applied stimulation set.

38. The method of claim 37, further comprising repeating the delivering, capturing, measuring, and determining for each stimulation set of the plurality of stimulation sets, to thereby determine a quality metric for each stimulation set of the plurality of stimulation sets.

39. The method of claim 38, further comprising selecting a stimulation set to be used as the applied stimulation set based on the determined quality metrics for each stimulation set.

40. The method of claim 39, further comprising using the selected stimulation set as the applied stimulation set.

41. A neurostimulation system comprising:
a multiple-stimset closed-loop neurostimulation device configured to controllably deliver neural stimuli according to a plurality of stimulation sets to a neural pathway of a patient so as to maintain a neural response intensity for at least an applied stimulation set of the plurality of stimulation sets at a corresponding target value; and
a processor configured to:
instruct the multiple-stimset closed-loop neurostimulation device to deliver a plurality of neural stimuli according to a first stimulation set of the plurality of stimulation sets;
receive a captured signal window corresponding to each delivered neural stimulus;
measure a characteristic of an evoked neural response in each captured signal window; and
determine a quality metric for the first stimulation set from the measured characteristics.

42. The neurostimulation system of claim 41, wherein the processor is configured to determine the quality metric for the first stimulation set from the measured characteristics by computing an independent metric for the first stimulation set.

43. The neurostimulation system of claim 41, wherein the processor is configured to determine the quality metric for the first stimulation set from the measured characteristics by computing a representativeness metric for the first stimulation set.

44. The neurostimulation system of claim 41, wherein the quality metric of the first stimulation set is indicative of the suitability of the first stimulation set to be used as the applied stimulation set.

45. The neurostimulation system of claim 44, wherein the processor is further configured to repeat the instructing, receiving, measuring, and determining, to thereby obtain a quality metric for each stimulation set of the plurality of stimulation sets.

46. The neurostimulation system of claim 45, wherein the processor is further configured to select a stimulation set to be used as the applied stimulation set based on the determined quality metrics for each stimulation set.

47. The neurostimulation system of claim 46, wherein the processor is further configured to program the multiple-stimset closed-loop device to use the selected stimulation set as the applied stimulation set.

48. The neurostimulation system of claim 41, wherein the processor is part of the neurostimulation device.

49. The neurostimulation system of claim 41, further comprising an external computing device in communication with the neurostimulation device.

50. The neurostimulation system of claim 49, wherein the processor is part of the external computing device.
